(19) United States
(12) Patent Application Publication
(10) Pub. No.: US 2026/0232535 A1
(43) Pub. Date: Aug. 13, 2026

(54) DRUG DELIVERY SYSTEM

(71) Applicant: BUDDHIST TZU CHI MEDICAL FOUNDATION, Hualien City (TW)

(72) Inventors: Ren-Jun Hsu, Taipei City (TW); Hui-Chun Chung, Hualien City (TW); Wen-Lin Hsu, Hualien City (TW); Min-We Hung, Hsinchu City (TW); Jung-Jung Nien, Hsinchu City (TW); Wen-Ning Chuang, Hsinchu City (TW); Chun-Ming Chang, New Taipei City (TW); Chuan-Yi Lin, Miaoli County (TW)

(21) Appl. No.: 19/535,084

(22) Filed: Feb. 10, 2026

(30) Foreign Application Priority Data
Feb. 10, 2025 (TW) 114104838

(51) Int. Cl. A61J 7/04; A61J 7/00

(52) U.S. Cl. CPC A61J 7/0454; A61J 7/0084

(57) ABSTRACT

Provided is a drug delivery system, including a plurality of drug boxes, mobile storage equipment, and fixed storage equipment. Each of the mobile storage equipment and the fixed storage equipment can accommodate and lock at least one of the plurality of drug boxes. When the mobile storage equipment confirms a user identity corresponding to a received first authentication information and confirms that a first generation time of the first authentication information is within a set period of time, the mobile storage equipment unlocks the at least one of the plurality of drug boxes accommodated therein. When the fixed storage equipment confirms a user identity corresponding to a received second authentication information and confirms that a second generation time of the second authentication information is within a set period of time, the fixed storage equipment unlocks the at least one of the plurality of drug boxes accommodated therein.

### CROSS-REFERENCE TO RELATED APPLICATION

Priority is claimed on Taiwanese Patent Application No. 114104838, filed Feb. 10, 2025, the content of which is incorporated herein by reference.

### BACKGROUND

### 1. Technical Field

The present disclosure relates to a drug delivery system, and more particularly to a drug delivery system capable of ensuring safety in drug delivery and reducing a burden on nursing staff.

### 2. Description of Related Art

In a typical medication dispensing and delivery management workflow commonly used in hospitals, a hospital pharmacist first dispenses medications according to each patient’s prescription. Next, a nursing assistant transports, in batches, drug boxes containing the dispensed medications to respective nursing stations. Thereafter, a nurse distributes the drug boxes placed at the nursing stations one by one to each patient room to provide corresponding patient medication.

However, for nurses, frequently distributing drug boxes to each patient room consumes substantial time and manpower and is likely to affect the performance of other important nursing duties, thereby imposing a burden on the nurses. Further, conventional medication dispensing and delivery management workflows do not provide adequate drug delivery safety measures, such that risks of theft or misuse of medications may exist during delivery.

Accordingly, there is a need to provide a drug delivery system capable of effectively addressing the foregoing issues, which remains an urgent problem to be solved.

### SUMMARY

The present disclosure provides a drug delivery system capable of ensuring drug delivery safety and reducing a burden on nursing staff.

In at least one embodiment, the drug delivery system of the present disclosure comprises a plurality of drug boxes, mobile storage equipment, and fixed storage equipment. Each of the drug boxes comprises a box body, a lid body, and a lid locking member. An accommodating space is formed in the box body. The lid body is configured to be coupled to the box body to close the accommodating space. The lid locking member is configured to lock the lid body to keep the lid body closing the accommodating space. The mobile storage equipment comprises a first frame and a first input unit and a first control unit disposed on the first frame. The first frame comprises a plurality of first compartments and a plurality of first locking mechanisms. Each of the first compartments is configured to accommodate any one of the drug boxes. Each of the first locking mechanisms is disposed corresponding to each of the first compartments. Each of the first locking mechanisms is configured to lock the drug box accommodated in the corresponding first compartment. The first input unit is configured to receive first authentication information. The first authentication information comprises first identity information and a first generation time of the first identity information. When the first control unit confirms that the first identity information corresponds to a user identity and that the first generation time is within a set period of time, the first control unit drives at least one of the plurality of first locking mechanisms to release locking of at least one of the plurality of drug boxes accommodated with respect to at least one of the plurality of first compartments. The fixed storage equipment comprises a second frame and a second input unit and a second control unit disposed on the second frame. The second frame comprises at least one second compartment and at least one second locking mechanism. Each of the second compartments is configured to accommodate any one of the drug boxes. Each of the second locking mechanisms is disposed corresponding to each of the second compartments. Each of the second locking mechanisms is configured to lock the drug box accommodated in the corresponding second compartment. The second input unit is configured to receive second authentication information. The second authentication information comprises second identity information and a second generation time of the second identity information. When the second control unit confirms that the second identity information corresponds to the user identity and that the second generation time is within a set period of time, the second control unit drives at least one of the at least one second locking mechanism to release locking of at least one of the plurality of drug boxes accommodated with respect to at least one of the at least one second compartment.

In at least one embodiment of the present disclosure, the first frame further comprises a plurality of first lid unlocking mechanisms, and each of the first lid unlocking mechanisms is disposed corresponding to each of the first compartments. When the user identity is confirmed to have medication dispensing permission, the first control unit drives the plurality of first locking mechanisms to release locking of at least one of the plurality of drug boxes accommodated with respect to at least one of the plurality of first compartments, and the first control unit drives the plurality of first lid unlocking mechanisms to unlock the lid locking member of at least one of the plurality of drug boxes accommodated in at least one of the plurality of first compartments.

In at least one embodiment of the present disclosure, the second frame further comprises a plurality of second lid unlocking mechanisms, and each of the second lid unlocking mechanisms is disposed corresponding to each of the second compartments. When the user identity is confirmed to have patient medication permission, the second control unit drives the at least one second locking mechanism to release locking of at least one of the plurality of drug boxes accommodated with respect to at least one of the at least one second compartment, and the second control unit drives the plurality of second lid unlocking mechanisms to unlock the lid locking member of at least one of the plurality of drug boxes accommodated in at least one of the plurality of second compartments.

In at least one embodiment of the present disclosure, after locking of at least one of the plurality of drug boxes accommodated in at least one of the plurality of first compartments is released, the at least one of the plurality of drug boxes accommodated in at least one of the plurality of first compartments is enabled by at least one of the corresponding plurality of first locking mechanisms to at least partially exit the at least one of the corresponding plurality of first compartments.

In at least one embodiment of the present disclosure, after locking of at least one of the plurality of drug boxes accommodated in at least one of the plurality of second compartments is released, the at least one of the plurality of drug boxes accommodated in at least one of the plurality of second compartments is enabled by at least one of the corresponding plurality of second locking mechanisms to at least partially exit the at least one of the corresponding plurality of second compartments.

In at least one embodiment of the present disclosure, the mobile storage equipment further comprises a first power supply unit. The first power supply unit is disposed in the first frame and is configured to supply power to the first control unit. The fixed storage equipment further comprises a second power supply unit. The second power supply unit is disposed in the second frame and is configured to supply power to the second control unit.

In at least one embodiment of the present disclosure, at least one of the first input unit and the second input unit is a two-dimensional code scanning device.

In at least one embodiment, the drug delivery system of the present disclosure further comprises a portable electronic device configured to generate first authentication information or second authentication information.

In at least one embodiment of the present disclosure, the portable electronic device comprises a processing module. The processing module is configured to generate a login interface to receive login information of any user and confirm the user identity corresponding to the user, and configured to correspondingly generate first authentication information or second authentication information according to the user identity.

In at least one embodiment of the present disclosure, when the processing module confirms that the user identity has drug dispensing permission, the processing module is configured to generate a compartment number input interface to receive compartment number information, and the processing module is configured to correspondingly generate first authentication information according to the user identity and the compartment number information, wherein the first authentication information further comprises the compartment number information.

In at least one embodiment of the present disclosure, when the processing module confirms that the user identity has drug dispensing permission or patient medication permission, the processing module is configured to generate a compartment number input interface to receive compartment number information, and the processing module is configured to correspondingly generate second authentication information according to the user identity and the compartment number information, wherein the second authentication information further comprises the compartment number information.

### BRIEF DESCRIPTION OF THE DRAWINGS

The present disclosure can be more fully understood by reading the following description of embodiments with reference to the accompanying drawings.

FIG. 1 is a schematic diagram of the drug delivery system of the present disclosure.

FIG. 2 is a side perspective schematic view of a drug box of the drug delivery system of the present disclosure.

FIG. 3 is a front view of the mobile storage equipment of the drug delivery system of the present disclosure.

FIG. 4 is a side perspective schematic view of the mobile storage equipment of the drug delivery system of the present disclosure without accommodating the drug box.

FIG. 5 is a side perspective schematic view of the mobile storage equipment of the drug delivery system of the present disclosure after accommodating the drug box.

FIG. 6 is a front view of the fixed storage equipment of the drug delivery system of the present disclosure.

FIG. 7 is a side perspective schematic view of the fixed storage equipment of the drug delivery system of the present disclosure without accommodating the drug box.

FIG. 8 is a side perspective schematic view of the fixed storage equipment of the drug delivery system of the present disclosure after accommodating the drug box.

FIG. 9 is a block diagram of constituent modules of the portable electronic device of the drug delivery system of the present disclosure.

FIG. 10 is a schematic diagram of an unlocking process of the plurality of drug boxes and the mobile storage equipment of the drug delivery system of the present disclosure.

FIG. 11 is a schematic diagram of an unlocking process of a single drug box and the fixed storage equipment of the drug delivery system of the present disclosure.

### DETAILED DESCRIPTION OF THE EMBODIMENTS

Since various aspects and embodiments are illustrative rather than limiting, a person having ordinary skill in the art, after reading this specification, may appreciate that other aspects and embodiments are possible without departing from the scope of the present disclosure. The following detailed description, together with the claims, will further highlight the features and advantages of the embodiments.

As used herein, the terms “a” or “an” are used to describe components and assemblies described herein. This is merely for convenience of description and provides a general meaning to the scope of the present disclosure. Accordingly, unless clearly indicated otherwise, such description is to be understood to include one or at least one, and the singular also includes the plural.

As used herein, the terms such as “first” and “second” and similar ordinal terms are primarily used to distinguish or refer to the same or similar components or structures and do not necessarily imply an order of such components or structures in space or time. It should be understood that, in certain situations or aspects, the ordinal terms may be used interchangeably without affecting implementation of the present disclosure.

As used herein, the terms “include,” “have,” “comprise,” or any other similar terms are intended to cover a non-exclusive inclusion. For example, a component or structure that contains a plurality of elements is not limited only to those elements listed herein but may include other elements not expressly listed yet ordinarily inherent to that component or structure.

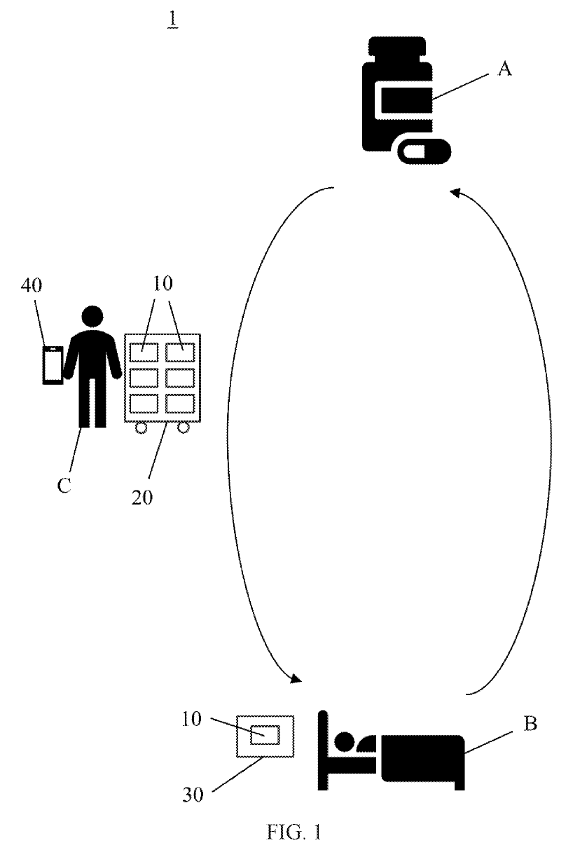

Referring to FIG. 1, FIG. 1 is a system schematic diagram of the drug delivery system 1 of the present disclosure. As shown in FIG. 1, the drug delivery system 1 of the present disclosure is primarily applied in medical institutions and is used by user C, such as a pharmacist, a nurse, and a nursing assistant, who divide responsibilities among themselves to complete a drug delivery and patient medication workflow from a pharmacy-side terminal A to a ward-side terminal B. Referring to FIG. 1, FIG. 1 is a schematic diagram of the drug delivery system 1 of the present disclosure. As shown in FIG. 1, the drug delivery system 1 of the present disclosure comprises a plurality of drug boxes 10, mobile storage equipment 20, and fixed storage equipment 30. In some embodiments of the present disclosure, the mobile storage equipment 20 can accommodate and lock at least one of the plurality of drug boxes 10 to facilitate medication dispensing by a pharmacist at the pharmacy-side terminal A and to facilitate safe delivery of medications by a nursing assistant from the pharmacy-side terminal A to the ward-side terminal B. In some embodiments of the present disclosure, the fixed storage equipment 30 can be fixedly disposed corresponding to each patient bed at each ward-side terminal B, and the fixed storage equipment 30 can accommodate and lock at least one of the plurality of drug boxes 10 to facilitate replacement of old and new drug boxes 10 by a nursing assistant at the ward-side terminal B and to facilitate safe patient medication by a nurse at the ward-side terminal B.

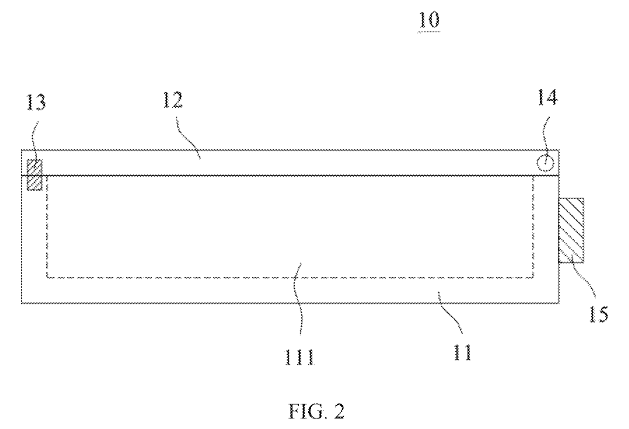

Referring to FIG. 2, FIG. 2 is a side perspective schematic view of a drug box 10 of the drug delivery system 1 of the present disclosure. As shown in FIGS. 1 and 2, each drug box 10 of the drug delivery system 1 of the present disclosure is used to store medication required by a patient. In at least one embodiment of the present disclosure, the drug box 10 comprises a box body 11, a lid body 12, and a lid locking member 13. An accommodating space 111 for storing medications is formed in the box body 11. The lid body 12 can be coupled to the box body 11, such that the lid body 12 closes the accommodating space 111. The lid locking member 13 can be optionally disposed on the box body 11 or the lid body 12. The lid locking member 13 locks the lid body 12 to the box body 11 to keep the lid body 12 closing the accommodating space 111. In some embodiments of the present disclosure, the box body 11 and the lid body 12 can be made of a relatively rigid metal or plastic material. In some embodiments of the present disclosure, the lid locking member 13 can be an electronically controlled lock, such as an electromagnetic lock; however, the present disclosure is not limited thereto.

For example, in some embodiments of the present disclosure, one side of the lid body 12 can be connected to the box body 11 by a hinge member 14, such that the lid body 12 is rotatable relative to the box body 11 in a flip-top configuration. When the lid body 12 is not yet locked by the lid locking member 13, a user can apply a force to the other side of the lid body 12 (i.e., the side opposite to the side hinge-connected to the box body 11) to move the lid body 12 relative to the box body 11 to an open position, thereby allowing medications to be removed from the accommodating space 111 of the box body 11 or to be stored in the accommodating space 111 of the box body 11. Alternatively, the lid body 12 can be moved relative to the box body 11 to a closed position, so that the lid body 12 is locked by the lid locking member 13 and the lid body 12 is kept closing the accommodating space 111. Since the drug box 10 per se does not have a power source, after the box body 11 and the lid body 12 of the drug box 10 are locked by the lid locking member 13, the user cannot directly unlock the lid locking member 13 under normal conditions, thereby providing storage security for the contents (for example, medications). To address medication access needs under emergency conditions, the lid locking member 13 of the drug box 10 can be additionally designed with a manual unlocking mechanism to allow the user to manually release locking of the lid body 12 by the lid locking member 13.

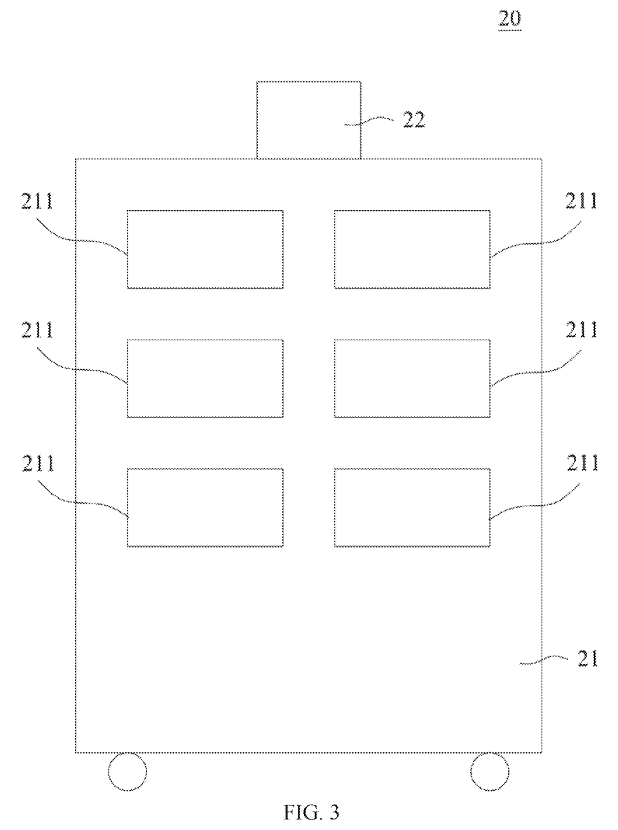

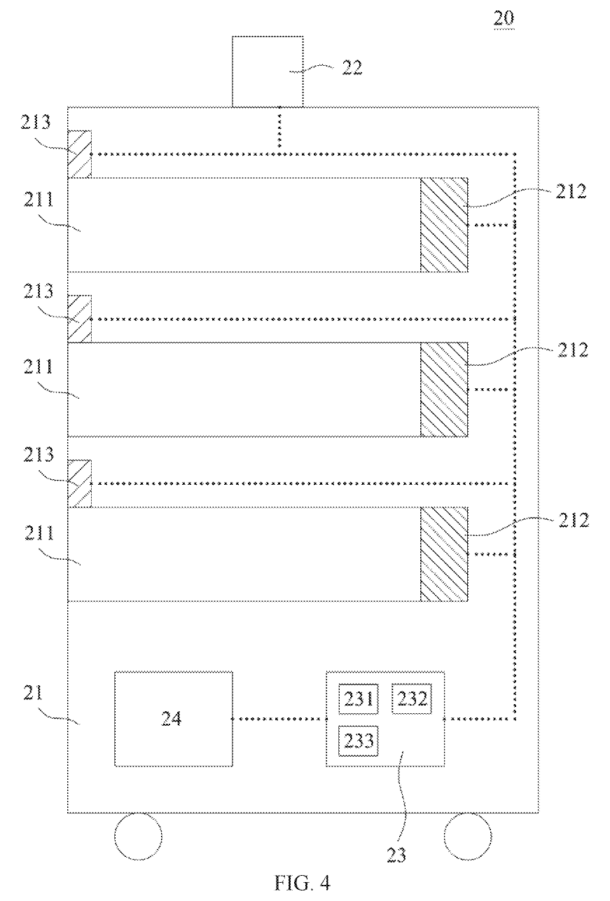

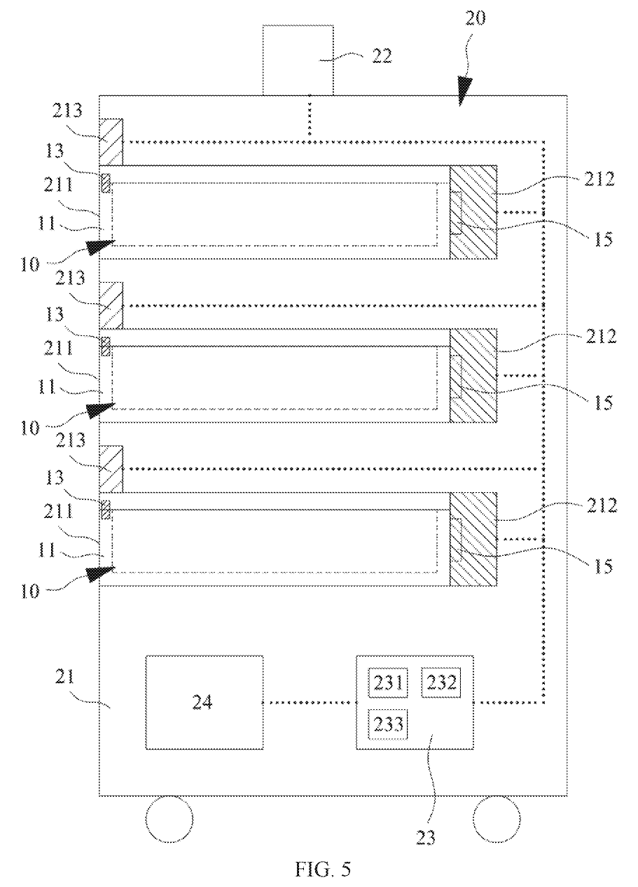

Referring also to FIGS. 3 to 5, FIG. 3 is a front view of the mobile storage equipment 20 of the drug delivery system 1 of the present disclosure; FIG. 4 is a side perspective schematic view of the mobile storage equipment 20 of the drug delivery system 1 of the present disclosure without accommodating the drug box 10; and FIG. 5 is a side perspective schematic view of the mobile storage equipment 20 of the drug delivery system 1 of the present disclosure after accommodating the drug box 10.

As shown in FIGS. 3 to 5, the mobile storage equipment 20 of the present disclosure can be used to store or transport one or more drug boxes 10. In at least one embodiment of the present disclosure, the mobile storage equipment 20 comprises a first frame 21, a first input unit 22, and a first control unit 23, and the first input unit 22 and the first control unit 23 are disposed on the first frame 21. In some embodiments of the present disclosure, the first frame 21 comprises a plurality of first compartments 211 and a plurality of first locking mechanisms 212, and each of the first locking mechanisms 212 is disposed corresponding to each of the first compartments 211. In some embodiments of the present disclosure, each of the first compartments 211 can accommodate any one of the drug boxes 10, and each of the first locking mechanisms 212 is configured to lock the drug box 10 accommodated in the corresponding first compartment 211. That is to say, after any one of the drug boxes 10 is placed into any one of the first compartments 211 of the first frame 21, the drug box 10 can be locked by the first locking mechanism 212 corresponding to the first compartment 211 to prevent the drug box 10 from being arbitrarily removed. In some embodiments of the present disclosure, each of the first compartments 211 is correspondingly assigned a specific first compartment number.

In some embodiments of the present disclosure, the box body 11 of each of the drug boxes 10 can further be provided with a corresponding locking structure 15 (as shown in FIGS. 2 or 5) to cooperate with the first locking mechanism 212 to effectively perform a locking function for the drug box 10. In such embodiments, an installation position of the first locking mechanism 212 can correspond to an installation position of the corresponding locking structure 15 of the drug box 10 accommodated in the first compartment 211. In some embodiments of the present disclosure, each of the first locking mechanisms 212 can be an electronically controlled lock, such as an electromagnetic lock, and the corresponding locking structure 15 of the box body 11 of the drug box 10 can be a protrusion, a recess, or another component that can cooperate with the electronically controlled lock for locking and unlocking; however, the present disclosure is not limited thereto.

In some embodiments of the present disclosure, the number of the plurality of first compartments 211 and the number of the plurality of first locking mechanisms 212 can vary depending on design requirements. For example, as shown in FIGS. 1 and 3, the number of the plurality of first compartments 211 and the number of the plurality of first locking mechanisms 212 are each six, such that the mobile storage equipment 20 can simultaneously store or transport six drug boxes 10; however, the present disclosure is not limited thereto. In some embodiments of the present disclosure, the first frame 21 can be made of a relatively rigid metal or plastic material, and a bottom portion of the first frame 21 can be provided with a plurality of wheels to facilitate movement. Accordingly, the mobile storage equipment 20 can be used as a mobile medication cart for medication dispensing by pharmacists and medication distribution by nursing assistants.

In some embodiments of the present disclosure, the first input unit 22 is configured to receive first authentication information entered by a user. In some embodiments of the present disclosure, the first authentication information at least comprises first identity information and a first generation time of the first identity information, wherein the first identity information is used to reflect an identity of the user, and the first generation time is used to reflect a time point at which the first identity information is generated. In some embodiments of the present disclosure, the first authentication information can further comprise first compartment number information, and the first compartment number information corresponds to a first compartment number corresponding to one of the plurality of first compartments 211 of the mobile storage equipment 20. In some embodiments of the present disclosure, the first input unit 22 can be a two-dimensional code (a quick response code) scanning device, and the first authentication information can be presented in the form of a two-dimensional code; however, the present disclosure is not limited thereto.

In some embodiments of the present disclosure, the first control unit 23 is electrically connected to the first input unit 22 and the plurality of first locking mechanisms 212, and the first control unit 23 is configured to determine, based on the first authentication information received by the first input unit 22, whether to drive at least one of the plurality of first locking mechanisms 212 of the first frame 21 to perform an unlocking operation. In some embodiments of the present disclosure, the first control unit 23 can be implemented as hardware (for example, a processor, a circuit board, a memory, a timer, a network chip, and other elements), software (for example, computer software, firmware, or an application), or a combination of the foregoing hardware and software. In some embodiments of the present disclosure, the first control unit 23 at least comprises an identity recognition module 231, a timing module 232, and an unlocking module 233, and these modules are electrically connected to each other. In some embodiments of the present disclosure, the identity recognition module 231 is configured to confirm the user identity corresponding to the first identity information of the first authentication information and whether the user identity has corresponding unlocking permission. The timing module 232 is configured to confirm whether a time difference between the first generation time of the first identity information and a current time point is within the set period of time. In some embodiments of the present disclosure, the set period of time can be between 30 seconds and 60 seconds, but the present disclosure is not limited thereto. The unlocking module 233 is configured to drive at least one of the plurality of first locking mechanisms 212 to perform the unlocking operation according to corresponding unlocking permission of the user identity. In some embodiments of the present disclosure, the first control unit 23 can perform identity recognition, compartment number matching, and time difference comparison based on data stored in the memory or external data obtained by connecting through a network to an external database.

In at least one embodiment of the present disclosure, when the first control unit 23 has confirmed the user identity corresponding to the first identity information in the first authentication information and has confirmed that the first generation time of the first identity information is within the set period of time, the first control unit 23 can drive at least one of the plurality of first locking mechanisms 212 according to different user identities (for example, driving only a single first locking mechanism 212 at a time, or driving a plurality of, or even all of, the first locking mechanisms 212 at a time) to release locking of at least one of the plurality of drug boxes 10 accommodated in at least one of the plurality of first compartments 211. In at least one embodiment of the present disclosure, to address access needs for the drug box 10 under emergency conditions, any one of the first locking mechanisms 212 of the first frame 21 can also be designed with a manual unlocking mechanism, such that a user can manually release locking of the drug box 10 by the first locking mechanism 212.

In some embodiments of the present disclosure, each of the first locking mechanisms 212 can be additionally provided with an ejection member (not shown) for the drug box 10, such as a push rod or a similar element. After locking of at least one of the plurality of drug boxes 10 accommodated in at least one of the plurality of first compartments 211 of the first frame 21 is released, by driving an ejection member of at least one of the corresponding plurality of first locking mechanisms 212, at least one of the plurality of drug boxes 10 accommodated in at least one of the plurality of first compartments 211 can be partially ejected from the at least one of the corresponding plurality of first compartments 211, thereby facilitating the user to remove at least one of the drug boxes 10.

In some embodiments of the present disclosure, the first frame 21 can further comprise a plurality of first lid unlocking mechanisms 213, and the first control unit 23 is electrically connected to the plurality of first lid unlocking mechanisms 213. In some embodiments of the present disclosure, each of the first lid unlocking mechanisms 213 is disposed corresponding to each of the first compartments 211, wherein an installation position of the first lid unlocking mechanism 213 can correspond to an installation position of the lid locking member 13 of the drug box 10 accommodated in the first compartment 211. In some embodiments of the present disclosure, when the user identity is confirmed to have medication dispensing permission, the first control unit 23 drives the plurality of first locking mechanisms 212 to release locking of at least one of the plurality of drug boxes 10 accommodated in at least one of the plurality of first compartments 211, and the first control unit 23 also drives the plurality of first lid unlocking mechanisms 213 to unlock the lid locking member 13 of at least one of the plurality of drug boxes 10 accommodated in at least one of the plurality of first compartments 211.

In some embodiments of the present disclosure, the mobile storage equipment 20 can further comprise a first power supply unit 24. In some embodiments of the present disclosure, the first power supply unit 24 is disposed in the first frame 21, and the first power supply unit 24 is electrically connected to the first control unit 23 to provide electrical power required for the first control unit 23 to perform corresponding functions. In some embodiments of the present disclosure, the first power supply unit 24 can be a rechargeable battery module, such as a lithium battery module; however, the present disclosure is not limited thereto.

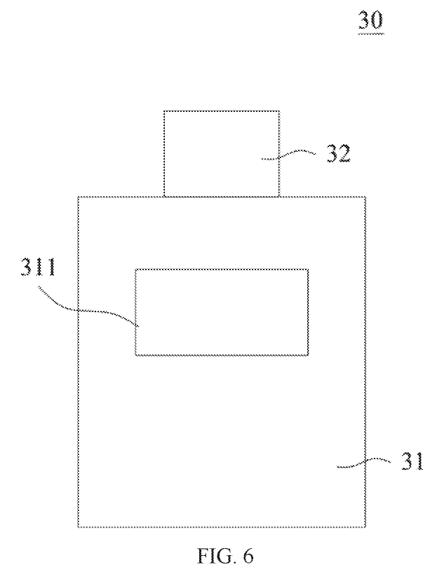

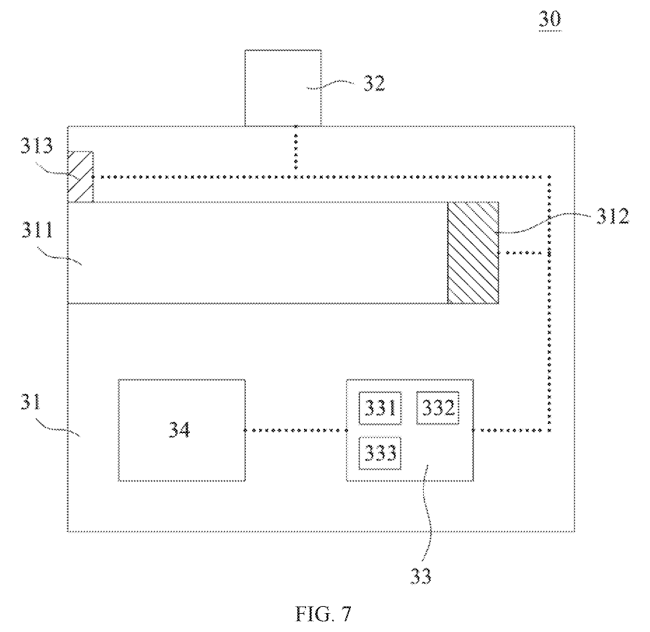

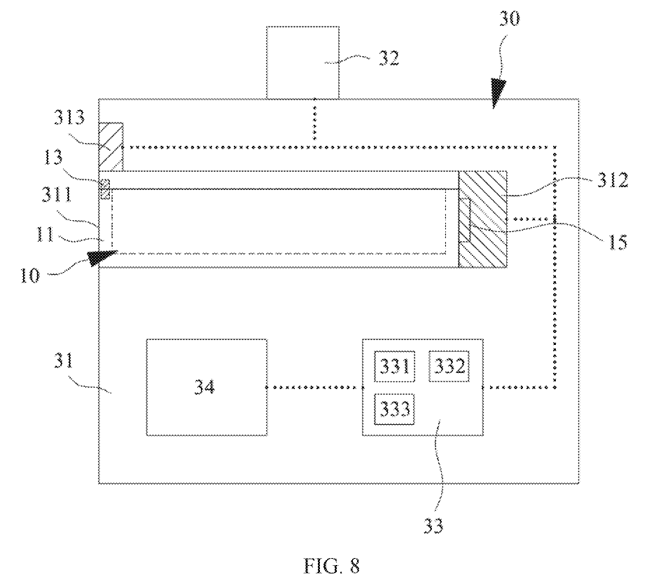

Referring also to FIGS. 6 to 8, FIG. 6 is a front view of the fixed storage equipment 30 of the drug delivery system 1 of the present disclosure; FIG. 7 is a side perspective schematic view of the fixed storage equipment 30 of the drug delivery system 1 of the present disclosure without accommodating the drug box 10; and FIG. 8 is a side perspective schematic view of the fixed storage equipment 30 of the drug delivery system 1 of the present disclosure after accommodating the drug box 10.

As shown in FIGS. 6 to 8, the fixed storage equipment 30 of the present disclosure can be used to store at least one drug box 10. In at least one embodiment of the present disclosure, the fixed storage equipment 30 comprises a second frame 31, a second input unit 32, and a second control unit 33, and the second input unit 32 and the second control unit 33 are disposed on the second frame 31. In some embodiments of the present disclosure, the second frame 31 comprises at least one second compartment 311 and at least one second locking mechanism 312, and each of the second locking mechanisms 312 is disposed corresponding to each of the second compartments 311. In some embodiments of the present disclosure, each of the second compartments 311 can accommodate any one of the drug boxes 10, and each of the second locking mechanisms 312 is configured to lock the drug box 10 accommodated in the corresponding second compartment 311. That is to say, after any one of the drug boxes 10 is placed into any one of the second compartments 311 of the second frame 31, the drug box 10 can be locked by the second locking mechanism 312 corresponding to the second compartment 311 to prevent the drug box 10 from being arbitrarily removed. In some embodiments of the present disclosure, when the at least one second compartment 311 comprises a plurality of second compartments 311, each of the second compartments 311 can also be correspondingly assigned a specific second compartment number.

In some embodiments of the present disclosure, the corresponding locking structure 15 provided on the box body 11 of each of the foregoing drug boxes 10 can likewise cooperate with the second locking mechanism 312 to effectively perform a locking function for the drug box 10. In such embodiments, an installation position of the second locking mechanism 312 can correspond to an installation position of the corresponding locking structure 15 of the drug box 10 accommodated in the second compartment 311. In some embodiments of the present disclosure, each of the second locking mechanisms 312 can be an electronically controlled lock, such as an electromagnetic lock, and the corresponding locking structure 15 of the box body 11 of the drug box 10 can be a protrusion, a recess, or another component that can cooperate with the electronically controlled lock for locking and unlocking; however, the present disclosure is not limited thereto.

In some embodiments of the present disclosure, the number of the at least one second compartment 311 and the number of the at least one second locking mechanism 312 can vary depending on design requirements. For example, the number of the at least one second compartment 311 and the number of the at least one second locking mechanism 312 can each be one, such that the fixed storage equipment 30 can store at least one drug box 10; however, the present disclosure is not limited thereto. In some embodiments of the present disclosure, the second frame 31 can be made of a relatively rigid metal or plastic material. Accordingly, the fixed storage equipment 30 can be used as a medication cabinet for medication distribution by nursing assistants and for patient medication by nurses, and the fixed storage equipment 30 can be fixedly disposed beside a patient bed at each ward-side terminal B.

In some embodiments of the present disclosure, the second input unit 32 is configured to receive second authentication information entered by a user. In some embodiments of the present disclosure, the second authentication information at least comprises second identity information and a second generation time of the second identity information, wherein the second identity information is used to reflect an identity of the user, and the second generation time is used to reflect a time point at which the second identity information is generated. In some embodiments of the present disclosure, the second authentication information can further comprise second compartment number information, and the second compartment number information corresponds to a second compartment number corresponding to one of the at least one second compartment 311 of the fixed storage equipment 30. In some embodiments of the present disclosure, the second input unit 32 can be a two-dimensional code scanning device, and the second authentication information can be presented in the form of a two-dimensional code; however, the present disclosure is not limited thereto.

In some embodiments of the present disclosure, the second control unit 33 is electrically connected to the second input unit 32 and the at least one second locking mechanism 312, and the second control unit 33 is configured to determine, based on the second authentication information received by the second input unit 32, whether to drive at least one of the at least one second locking mechanism 312 of the second frame 31 to perform an unlocking operation. In some embodiments of the present disclosure, the second control unit 33 can be implemented as hardware (for example, a processor, a circuit board, a memory, a timer, a network chip, and other elements), software (for example, computer software, firmware, or an application), or a combination of the foregoing hardware and software. In at least one embodiment of the present disclosure, the second control unit 33 at least comprises an identity recognition module 331, a timing module 332, and an unlocking module 333, and these modules are electrically connected to each other. In some embodiments of the present disclosure, the identity recognition module 331 is configured to confirm the user identity corresponding to the second identity information of the second authentication information and whether the user identity has corresponding unlocking permission. The timing module 332 is configured to confirm whether a time difference between the second generation time of the second identity information and a current time point is within the set period of time. In some embodiments of the present disclosure, the set period of time can be between 30 seconds and 60 seconds, but the present disclosure is not limited thereto. The unlocking module 333 is configured to drive at least one of the at least one second locking mechanism 312 to perform the unlocking operation according to corresponding unlocking permission of the user identity. In some embodiments of the present disclosure, the second control unit 33 can perform identity recognition, compartment number matching, and time difference comparison based on data stored in the memory or external data obtained by connecting through a network to an external database.

In at least one embodiment of the present disclosure, when the second control unit 33 has confirmed the user identity corresponding to the second identity information in the second authentication information and has confirmed that the second generation time of the second identity information is within the set period of time, the second control unit 33 can drive at least one of the at least one second locking mechanism 312 according to different user identities (for example, driving only a single second locking mechanism 312 at a time, or driving a plurality of, or even all of, the second locking mechanisms 312 at a time) to release locking of at least one of the plurality of drug boxes 10 accommodated in at least one of the at least one second compartment 311. In some embodiments of the present disclosure, to address access needs for the drug box 10 under emergency conditions, any one of the second locking mechanisms 312 of the second frame 31 can also be designed with a manual unlocking mechanism, such that a user can manually release locking of the drug box 10 by the second locking mechanism 312.

In some embodiments of the present disclosure, each of the second locking mechanisms 312 can be additionally provided with an ejection member (not shown) for the drug box 10, such as a push rod or a similar element. After locking of at least one of the plurality of drug boxes 10 accommodated in at least one of the plurality of second compartments 311 of the second frame 31 is released, by driving an ejection member of at least one of the corresponding plurality of second locking mechanisms 312, at least one of the plurality of drug boxes 10 accommodated in at least one of the plurality of second compartments 311 can be partially ejected from at least one of the corresponding plurality of second compartments 311, thereby facilitating the user to remove at least one of the drug boxes 10.

In some embodiments of the present disclosure, the second frame 31 can further comprise at least one second lid unlocking mechanism 313, and the second control unit 33 is electrically connected to the at least one second lid unlocking mechanism 313. In some embodiments of the present disclosure, each of the second lid unlocking mechanisms 313 is disposed corresponding to each of the second compartments 311, wherein an installation position of the second lid unlocking mechanism 313 can correspond to an installation position of the lid locking member 13 of the drug box 10 accommodated in the second compartment 311. In some embodiments of the present disclosure, when the user identity is confirmed to have patient medication permission, the second control unit 33 drives at least one second locking mechanism 312 to release locking of at least one of the at least one drug box 10 accommodated in at least one of the at least one second compartment 311, and the second control unit 33 also drives the at least one second lid unlocking mechanism 313 to unlock the lid locking member 13 of at least one of the plurality of drug boxes 10 accommodated in at least one of the at least one second compartment 311.

In at least one embodiment of the present disclosure, the fixed storage equipment 30 can further comprise a second power supply unit 34. In some embodiments of the present disclosure, the second power supply unit 34 is disposed in the second frame 31, and the second power supply unit 34 is electrically connected to the second control unit 33 to provide electrical power required for the second control unit 33 to perform corresponding functions. In some embodiments of the present disclosure, the second power supply unit 34 can be a power circuit module connectable to mains power (including a transformer and a power cord, among others); however, the present disclosure is not limited thereto.

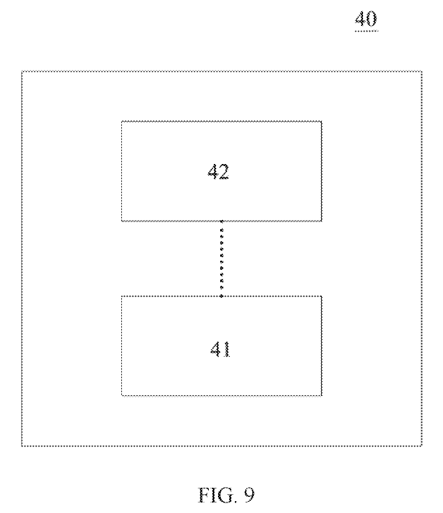

Referring to FIG. 9, FIG. 9 is a block diagram of constituent modules of the portable electronic device 40 of the drug delivery system 1 of the present disclosure. As shown in FIGS. 1 and 9, in at least one embodiment, the drug delivery system 1 of the present disclosure further comprises the portable electronic device 40, which is configured to generate the first authentication information or the second authentication information. In some embodiments of the present disclosure, the portable electronic device 40 can be a smartphone, a tablet computer, or a similar electronic operating device, and the present disclosure is not limited thereto. In some embodiments of the present disclosure, the portable electronic device 40 comprises a processing module 41 and a display module 42, wherein the processing module 41 is configured to generate a login interface that is to be displayed by the display module 42, such that any user C can enter login information via the login interface. In some embodiments of the present disclosure, the processing module 41 can receive login information of any user C, confirm a user identity corresponding to the user based on the login information, and then correspondingly generate the first authentication information or the second authentication information based on the confirmed user identity. Then, the first authentication information or the second authentication information is displayed on the display module 42. In some embodiments of the present disclosure, the first authentication information and the second authentication information can be presented in the form of a two-dimensional code, but the present disclosure is not limited thereto. In some embodiments of the present disclosure, the processing module 41 can be implemented as a combination of hardware (for example, a processor and a memory) and software (for example, an application), wherein the application can be built in the device or can be downloaded and installed via a network, and the login interface described above can be generated after the application is executed by the processor.

Further, in some embodiments of the present disclosure, when the processing module 41 confirms that the user identity has drug dispensing permission, the processing module 41 can further generate a compartment number input interface that is to be displayed by the display module 42, such that the user C can enter, via the compartment number input interface, compartment number information corresponding to any one of the first compartments 211. In some embodiments of the present disclosure, the processing module 41 can receive the compartment number information entered by the user C, and the processing module 41 can correspondingly generate the first authentication information or the second authentication information according to the user identity and the first compartment number information.

The drug delivery system 1 of the present disclosure can provide, according to different user identities corresponding to users, different unlocking permissions for the drug boxes stored in the mobile storage equipment 20 or the fixed storage equipment 30, and can also provide different unlocking permissions for the lid locking members 13 of the drug boxes 10 per se. That is to say, in the present disclosure, depending on the permissions provided, a first portion of users (for example, pharmacists having medication dispensing permission) can unlock, at one time, all of the drug boxes 10 temporarily stored in the mobile storage equipment 20 or the fixed storage equipment 30 and the lid locking members 13 thereof. A second portion of users (for example, nurses having patient medication permission) can unlock, at one time, only a single drug box 10 temporarily stored in the mobile storage equipment 20 or the fixed storage equipment 30 and the lid locking member 13 thereof. A third portion of users (for example, nursing assistants having medication distribution permission) can unlock, at one time, only a single drug box 10 temporarily stored in the mobile storage equipment 20 or the fixed storage equipment 30 but cannot unlock the lid locking member 13 of the drug box 10. Accordingly, by differentiating, based on different user identities, the unlocking permissions associated with medication delivery and storage equipment, the drug delivery system 1 of the present disclosure can effectively improve safety of medication delivery and patient medication.

The following describes, by way of example, various corresponding embodiments of the drug delivery system 1 of the present disclosure by illustrating application of the drug delivery system 1 of the present disclosure for users having different identities.

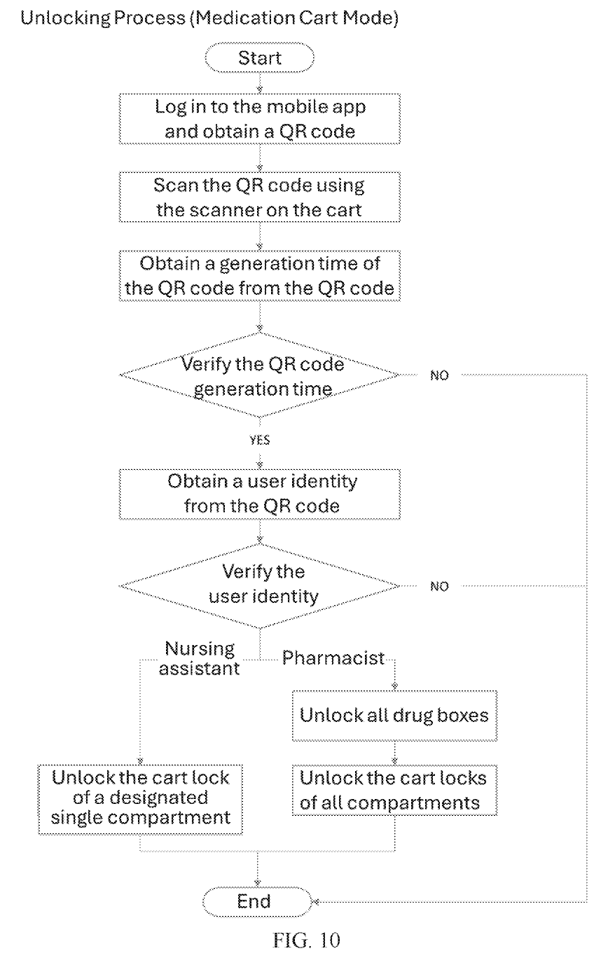

Referring also to FIGS. 1, 5, and 10, FIG. 10 is a schematic diagram of an unlocking process of the plurality of drug boxes 10 and the mobile storage equipment 20 of the drug delivery system 1 of the present disclosure. In the following description, the mobile storage equipment 20 can be a medication cart, and the mobile storage equipment 20 can comprise six first compartments 211 and correspondingly accommodate six drug boxes 10. The portable electronic device 40 can be a smartphone. As shown in FIGS. 1, 5, and 10, in at least one embodiment of the present disclosure, a user can enter login information through a user interface generated by the portable electronic device 40, and after receiving the login information of the user and confirming the user identity corresponding to the user, the portable electronic device 40 can generate and display the first authentication information according to the user identity.

In some embodiments of the present disclosure, when the portable electronic device 40 confirms that the user identity is a pharmacist, the portable electronic device 40 directly generates and displays the first authentication information based on the login information corresponding to the pharmacist identity. In such case, the first authentication information comprises first identity information and a first generation time of the first identity information. In some embodiments of the present disclosure, when the portable electronic device 40 confirms that the user identity is a nursing assistant, the portable electronic device 40 further displays, via the user interface, an input field for the user to further enter a first compartment number corresponding to a single first compartment 211 to be unlocked. Thereafter, the portable electronic device 40 generates and displays the first authentication information based on the login information corresponding to the nursing assistant identity and the first compartment number. In such case, the first authentication information comprises first identity information, the first generation time of the first identity information, and first compartment number information.

In some embodiments of the present disclosure, the user then brings the portable electronic device 40 close to the first input unit 22 of the mobile storage equipment 20, such that the first input unit 22 receives the first authentication information displayed by the portable electronic device 40. In some embodiments of the present disclosure, the first input unit 22 can transmit the received first authentication information to the first control unit 23, and the first control unit 23 confirms whether a time difference between the first generation time of the first identity information and a current time point is within the set period of time. If the time difference between the first generation time and the current time point is confirmed to exceed the set period of time, the unlocking process ends. If the time difference between the first generation time and the current time point is confirmed to be within the set period of time, the first control unit 23 further confirms the user identity corresponding to the first identity information of the first authentication information and whether the user identity has corresponding unlocking permission.

In some embodiments of the present disclosure, if the user identity is then confirmed to correspond to the first identity information of the first authentication information that does not have corresponding unlocking permission, the unlocking process ends. If the user identity is confirmed to correspond to the first identity information of the first authentication information that has corresponding unlocking permission, the first control unit 23 performs an unlocking operation for the mobile storage equipment 20 and the plurality of drug boxes 10 stored in the mobile storage equipment 20 according to the unlocking permission corresponding to the user identity. In some embodiments of the present disclosure, when the user identity is confirmed to be a pharmacist, the first control unit 23 transmits control signals to six first locking mechanisms 212 and six first lid unlocking mechanisms 213 corresponding to six first compartments 211, so as to drive the six first locking mechanisms 212 to unlock all six drug boxes 10 accommodated in the six first compartments 211 and also drive the six first lid unlocking mechanisms 213 to unlock all lid locking members 13 of the six drug boxes 10 accommodated in the six first compartments 211, thereby ending the entire unlocking process. Accordingly, all six drug boxes 10 stored in the mobile storage equipment 20 and the lid locking members 13 thereof are unlocked, thereby enabling the user (the pharmacist) to perform medication dispensing operations for each drug box 10. After the user completes medication dispensing for each drug box 10, the user can push each drug box 10 into the corresponding first compartment 211, such that the corresponding first locking mechanism 212 re-locks each drug box 10 and the lid body 12 of each drug box 10 is moved back to the closed position and locked by the lid locking member 13.

In some embodiments of the present disclosure, when the user identity is confirmed to be a nursing assistant, the first control unit 23 transmits a control signal, based on the first compartment number information, to the first locking mechanism 212 corresponding to a matching first compartment 211, so as to drive the first locking mechanism 212 to unlock the drug box 10 accommodated in the first compartment 211, thereby ending the entire unlocking process. In such case, the lid locking member 13 of the drug box 10 remains locked to the lid body 12 to ensure delivery safety of medication stored in the drug box 10. Accordingly, a single drug box 10 stored in the mobile storage equipment 20 is unlocked, thereby enabling the user (the nursing assistant) to remove the drug box 10 to perform subsequent medication distribution operations.

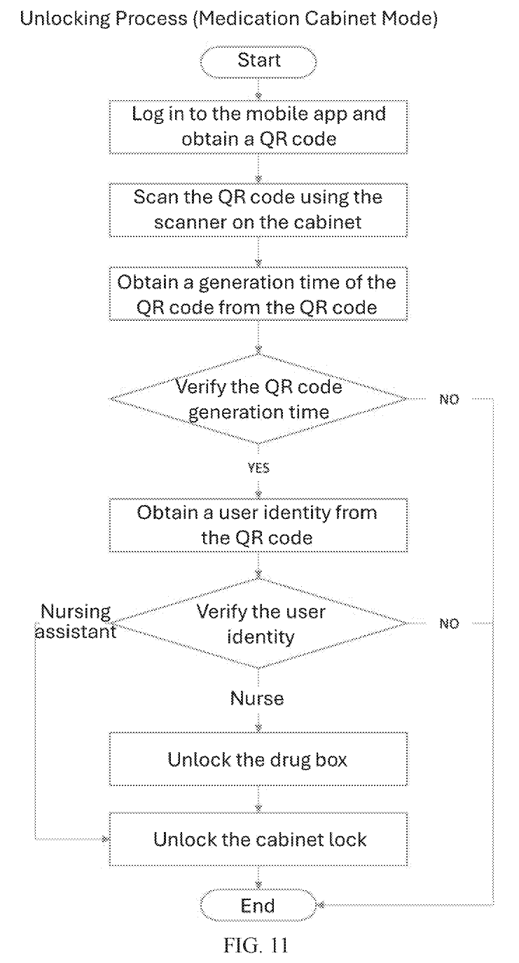

Referring also to FIGS. 1, 8, and 11, FIG. 11 is a schematic diagram of an unlocking process of a single drug box 10 and the fixed storage equipment 30 of the drug delivery system 1 of the present disclosure.

In the following description, the fixed storage equipment 30 can be a medication cabinet disposed in a ward, and the fixed storage equipment 30 comprises only one second compartment 311 and correspondingly accommodates one drug box 10. The portable electronic device 40 can be a smartphone. As shown in FIGS. 1, 8, and 11, in at least one embodiment of the present disclosure, a user can likewise enter login information through a user interface generated by the portable electronic device 40, and after receiving the login information of the user and confirming the user identity corresponding to the user, the portable electronic device 40 can generate and display the second authentication information according to the user identity.

In some embodiments of the present disclosure, given that the at least one second compartment 311 of the fixed storage equipment 30 comprises only a single second compartment 311, when the portable electronic device 40 confirms that the user identity is a nurse or a nursing assistant, the portable electronic device 40 directly generates and displays the second authentication information based on the login information corresponding to the nurse identity or the nursing assistant identity. In such case, the second authentication information comprises second identity information and a second generation time of the second identity information.

In some embodiments of the present disclosure, given that the at least one second compartment 311 of the fixed storage equipment 30 comprises a plurality of second compartments 311, when the portable electronic device 40 confirms that the user identity is a nurse or a nursing assistant, the portable electronic device 40 further displays, via the user interface, an input field for the user to further enter a second compartment number corresponding to a single second compartment 311 to be unlocked. Thereafter, the portable electronic device 40 generates and displays the second authentication information based on the login information corresponding to the nurse identity or the nursing assistant identity and the second compartment number. In such case, the second authentication information comprises second identity information, the second generation time of the second identity information, and second compartment number information.

In some embodiments of the present disclosure, the user then brings the portable electronic device 40 close to the second input unit 32 of the fixed storage equipment 30, such that the second input unit 32 receives the second authentication information displayed by the portable electronic device 40. In some embodiments of the present disclosure, the second input unit 32 can transmit the received second authentication information to the second control unit 33, and the second control unit 33 confirms whether a time difference between the second generation time of the second identity information and a current time point is within the set period of time. If the time difference between the second generation time and the current time point is confirmed to exceed the set period of time, the unlocking process ends. If the time difference between the second generation time and the current time point is confirmed to be within the set period of time, the second control unit 33 further confirms the user identity corresponding to the second identity information of the second authentication information and whether the user identity has corresponding unlocking permission.

In some embodiments of the present disclosure, if the user identity is then confirmed to correspond to the second identity information of the second authentication information that does not have corresponding unlocking permission, the unlocking process ends. If the user identity is confirmed to correspond to the second identity information of the second authentication information that has corresponding unlocking permission, the second control unit 33 performs an unlocking operation for the fixed storage equipment 30 and at least one drug box stored in the fixed storage equipment 30 according to the unlocking permission corresponding to the user identity. In some embodiments of the present disclosure, given that the at least one second compartment 311 of the fixed storage equipment 30 comprises only a single second compartment 311, when the user identity is confirmed to be a nurse, the second control unit 33 transmits a control signal to the second locking mechanism 312 and the second lid unlocking mechanism 313 corresponding to the second compartment 311, so as to drive the second locking mechanism 312 to unlock the drug box 10 accommodated in the second compartment 311 and also drive the second lid unlocking mechanism 313 to unlock the lid locking member 13 of the drug box 10 accommodated in the second compartment 311, thereby ending the entire unlocking process. In some embodiments of the present disclosure, given that the at least one second compartment 311 of the fixed storage equipment 30 comprises a plurality of second compartments 311, when the user identity is confirmed to be a nurse, the second control unit 33 transmits a control signal to the second locking mechanism 312 and the second lid unlocking mechanism 313 corresponding to the second compartment 311 matching the second compartment number information, so as to drive the second locking mechanism 312 to unlock the drug box 10 accommodated in the second compartment 311 and also drive the second lid unlocking mechanism 313 to unlock the lid locking member 13 of the drug box 10 accommodated in the second compartment 311, thereby ending the entire unlocking process. Accordingly, the drug box 10 stored in the fixed storage equipment 30 and the lid locking member 13 thereof are unlocked, thereby enabling the user (the nurse) to perform patient medication operations with respect to the drug box 10. After the user completes patient medication for the patient, the user can push the drug box 10 into the second compartment 311, such that the corresponding second locking mechanism 312 re-locks the drug box 10 and the lid body 12 of the drug box 10 is moved back to the closed position and locked by the lid locking member 13.

In some embodiments of the present disclosure, given that the at least one second compartment 311 of the fixed storage equipment 30 comprises only a single second compartment 311, when the user identity is confirmed to be a nursing assistant, the second control unit 33 transmits a control signal to the second locking mechanism 312 corresponding to the second compartment 311 to drive the second locking mechanism 312 to unlock the drug box 10 accommodated in the second compartment 311, thereby ending the entire unlocking process. In such case, the lid locking member 13 of the drug box 10 remains locked to the lid body 12. In some embodiments of the present disclosure, given that the at least one second compartment 311 of the fixed storage equipment 30 comprises a plurality of second compartments 311, when the user identity is confirmed to be a nursing assistant, the second control unit 33 transmits a control signal to the second locking mechanism 312 corresponding to the second compartment 311 matching the second compartment number information, so as to drive the second locking mechanism 312 to unlock the drug box 10 accommodated in the second compartment 311, thereby ending the entire unlocking process. In such case, the lid locking member 13 of the drug box 10 remains locked to the lid body 12. Accordingly, the drug box 10 stored in the fixed storage equipment 30 is unlocked, thereby enabling the user (the nursing assistant) to remove the drug box 10 to perform a subsequent drug box collection operation. After the user completes collection of the drug box 10, the user can place another new drug box 10 into the corresponding second compartment 311 for replacement, such that the corresponding second locking mechanism 312 locks the new drug box 10.

In summary, the drug delivery system 1 of the present disclosure can, according to different identities of a user, provide different unlocking permissions for drug boxes stored in the mobile storage equipment 20 or the fixed storage equipment 30 and can also provide different unlocking permissions for the lid locking members 13 of the drug boxes 10 per se. In the drug delivery system 1 of the present disclosure, after a pharmacist completes medication dispensing according to prescriptions, subsequent medication delivery tasks can be entirely performed by nursing assistants, who distribute medications directly from the pharmacy-side terminal A to storage at respective ward-side terminals B. A nurse only needs, when a patient is to take a medication, to retrieve the medication at the ward-side terminal B and verify the prescription, thereby assisting patient medication and further reducing a work burden on the nurse. In addition, the drug delivery system 1 of the present disclosure can effectively provide medication delivery safety during the medication delivery process and reduce a risk of medication theft.

The foregoing embodiments are inherently provided for purposes of illustration and are not intended to limit embodiments of the subject matter applied for or applications of such embodiments. Further, although at least one exemplary embodiment has been described in the foregoing embodiments, it should be understood that the present disclosure is applicable to numerous variations. It should likewise be understood that the embodiments described herein are not intended to limit, in any manner, the scope, use, or configuration of the claimed subject matter. Rather, the foregoing embodiments provide a convenient guide for a person having ordinary skill in the art to implement one or more of the described embodiments. Further, various changes may be made to the functions and arrangements of elements without departing from the scope as defined by the claims, and the claims encompass known equivalents as well as all foreseeable equivalents at the time of filing of this patent application.

What is claimed is:

1. A drug delivery system, comprising:
a plurality of drug boxes, each of the plurality of drug boxes comprising a box body, a lid body, and a lid locking member, wherein an accommodating space is formed in the box body, the lid body is configured to be coupled to the box body to close the accommodating space, and the lid locking member is configured to lock the lid body to keep the lid body closing the accommodating space;
mobile storage equipment comprising a first frame and a first input unit and a first control unit disposed on the first frame, wherein the first frame comprises a plurality of first compartments and a plurality of first locking mechanisms, each of the first compartments is configured to accommodate any one of the drug boxes, each of the first locking mechanisms is disposed corresponding to each of the first compartments, each of the first locking mechanisms is configured to lock the drug box accommodated in the corresponding first compartment, the first input unit is configured to receive first authentication information, the first authentication information comprises first identity information and a first generation time of the first identity information, and when the first control unit confirms that the first identity information corresponds to a user identity and that the first generation time is within a set period of time, the first control unit drives at least one of the plurality of first locking mechanisms to release locking of at least one of the plurality of drug boxes accommodated with respect to at least one of the plurality of first compartments; and
fixed storage equipment comprising a second frame and a second input unit and a second control unit disposed on the second frame, wherein the second frame comprises at least one second compartment and at least one second locking mechanism, each of the second compartments is configured to accommodate any one of the drug boxes, each of the second locking mechanisms is disposed corresponding to each of the second compartments, each of the second locking mechanisms is configured to lock the drug box accommodated in the corresponding second compartment, the second input unit is configured to receive second authentication information, the second authentication information comprises second identity information and a second generation time of the second identity information, and when the second control unit confirms that the second identity information corresponds to the user identity and that the second generation time is within a set period of time, the second control unit drives at least one of the at least one second locking mechanism to release locking of at least one of the plurality of drug boxes accommodated with respect to at least one of the at least one second compartment.

2. The drug delivery system of claim 1, wherein the first frame further comprises a plurality of first lid unlocking mechanisms, and each of the first lid unlocking mechanisms is disposed corresponding to each of the first compartments, and when the user identity is confirmed to have medication dispensing permission, the first control unit drives the plurality of first locking mechanisms to release locking of at least one of the plurality of drug boxes accommodated with respect to at least one of the plurality of first compartments, and the first control unit drives the plurality of first lid unlocking mechanisms to unlock the lid locking member of at least one of the plurality of drug boxes accommodated in at least one of the plurality of first compartments.

3. The drug delivery system of claim 1, wherein the second frame further comprises a plurality of second lid unlocking mechanisms, and each of the second lid unlocking mechanisms is disposed corresponding to each of the second compartments, and when the user identity is confirmed to have patient medication permission, the second control unit drives the at least one second locking mechanism to release locking of at least one of the plurality of drug boxes accommodated with respect to at least one of the at least one second compartment, and the second control unit drives the plurality of second lid unlocking mechanisms to unlock the lid locking member of at least one of the plurality of drug boxes accommodated in at least one of the plurality of second compartments.

4. The drug delivery system of claim 1, wherein after locking of at least one of the plurality of drug boxes accommodated in at least one of the plurality of first compartments is released, the at least one of the plurality of drug boxes accommodated in at least one of the plurality of first compartments is enabled by at least one of the corresponding plurality of first locking mechanisms to at least partially exit at least one of the corresponding plurality of first compartments.

5. The drug delivery system of claim 1, wherein after locking of at least one of the plurality of drug boxes accommodated in at least one of the plurality of second compartments is released, the at least one of the plurality of drug boxes accommodated in at least one of the plurality of second compartments is enabled by at least one of the corresponding plurality of second locking mechanisms to at least partially exit at least one of the corresponding plurality of second compartments.

6. The drug delivery system of claim 1, wherein the mobile storage equipment further comprises a first power supply unit disposed in the first frame and configured to supply power to the first control unit, and wherein the fixed storage equipment further comprises a second power supply unit disposed in the second frame and configured to supply power to the second control unit.

7. The drug delivery system of claim 1, further comprising a portable electronic device configured to generate the first authentication information or the second authentication information, wherein at least one of the first input unit and the second input unit is a two-dimensional code scanning device.

8. The drug delivery system of claim 7, wherein the portable electronic device comprises a processing module configured to generate a login interface to receive login information of any user and confirm the user identity corresponding to the user, and generate the first authentication information or the second authentication information correspondingly according to the user identity.

9. The drug delivery system of claim 8, wherein when the processing module confirms that the user identity has drug dispensing permission, the processing module is configured to generate a compartment number input interface to receive compartment number information, and the processing module is configured to correspondingly generate the first authentication information according to the user identity and the compartment number information, wherein the first authentication information further comprises the compartment number information.

10. The drug delivery system of claim 8, wherein when the processing module confirms that the user identity has drug dispensing permission or patient medication permission, the processing module is configured to generate a compartment number input interface to receive compartment number information, and the processing module is configured to correspondingly generate the second authentication information according to the user identity and the compartment number information, wherein the second authentication information further comprises the compartment number information.
